(19) United States
(12) Patent Application Publication
(10) Pub. No.: US 2002/0142337 A1
(43) Pub. Date: Oct. 3, 2002

(54) Hybrid gene libraries and uses thereof

(76) Inventor: David N. Edwards, Addison, TX (US)

Correspondence Address: BAKER & BOTTS, 30 ROCKEFELLER PLAZA, NEW YORK, NY, 10112

(21) Appl. No.: 10/071,136

(22) Filed: Feb. 6, 2002

Related U.S. Application Data
(60) Provisional application No. 60/279,788, filed on Mar. 29, 2001.

(51) Int. Cl. C12Q 1/68; C07H 21/04; C12N 15/74

(52) U.S. Cl. 435/6; 536/23.7; 435/320.1

(57) ABSTRACT

This invention relates to the construction and use of hybrid gene cDNA libraries. The vectors of such libraries each comprise a hybrid protein region in which cDNA is placed upstream of a sequence encoding a common peptide. The cDNA population inserted into the hybrid proteins is derived from an mRNA template population using random primers, thus providing better representation of the 5′ end than if poly-T primers were used. The vector lacks a start codon before the multiple cloning site or in the common peptide so that only cDNA inserts containing a start codon result in a hybrid protein.

This application claims priority to U.S. provisional application No. 60/279,788, filed Mar. 29, 2001.

### BACKGROUND OF THE INVENTION

This invention relates to the construction and use of hybrid gene cDNA libraries.

Complementary deoxyribonucleic acid, or cDNA libraries are collections of nucleotide sequences copied from messenger ribonucleic acid, or mRNA, isolated from specific organisms, tissues or cells. The usefulness of cDNA libraries stems from the fact that they ideally represent a collection of all, or at least most of, the mRNA molecules present in the starting material in a form that is more stable and easy to propagate than the mRNA itself. Hybrid gene libraries are a specific type in which the cDNAs are ligated into a cloning vector containing sequences encoding a peptide of defined composition, such that all cDNAs can be expressed in hybrid proteins in which the cDNA expression product is fused to the common peptide. This common peptide is the common peptide of the entire library. Hybrid gene libraries are especially useful for a variety of purposes:

Epitope or affinity tagging of gene products for detection/purification, if the common peptide is an epitope or affinity tag.

Subcellular targeting or secretion of library gene products, if the common peptide is a targeting or trafficking signal.

Tracer labeling of library gene products for detection, if the common peptide is a label traceable by luminescence, fluorescence or other methods.

Production of hybrid protein libraries for in vivo or in vitro screening, detection and quantitation of molecular interactions, using methods that may include yeast or other one-, two- or three-hybrid methods, fluorescence resonance energy transfer spectroscopy, affinity or immunoaffinity binding and other methods, which is referred to herein as “molecular interaction methods”, if the common peptide displays a biological activity dependent on one or more molecular interaction(s).

Traditional methods that utilize hybrid gene libraries for gene discovery are designed to yield results in a linear, gene-by-gene fashion. Those methods have been designed with the rationale that the discovery of a previously unknown gene is the starting point for research carried out by one or a few individuals. By contrast, more modern high-throughput automated methods allow the performance of certain assays at a scale hundreds of times that of older procedures. These methods permit, therefore, the performance of massive screens aimed at saturation of the system under study. The aim of modern high throughput gene screens is to discover all the possible genes involved in a specific area; that is, to “leave no stone unturned.” As it pertains to cDNA libraries, then, it becomes crucial to have total representation of mRNAs.

As currently constructed, cDNA libraries rarely achieve total representation, in large part because cDNA library clones frequently lack the 5′ end of the mRNA coding sequences. For example, FIG. 1 shows a common procedure for the construction of hybrid gene cDNA libraries. In FIG. 1a, mRNA molecules with a polyadenylated 3′ end are annealed to an oligo[dT] primer for first strand cDNA synthesis. FIG. 1b shows a consequence of the limitations of enzymatic in vitro cDNA synthesis: as reverse transcriptase moves along the mRNA to make the cDNA copy, it has a finite chance of “falling off” the mRNA at each step. The result is that each mRNA has a low probability of being copied to a significant extent with a higher probability of being copied as middle to short cDNAs.

Another consequence of priming the first strand at the 3′ end is that the cDNA will invariably contain the non-coding untranslated region or UTR found in mRNAs. When making hybrid gene cDNA libraries, as with molecular interaction methods, this dictates that the vector sequences encoding the common peptide must be 5′ to the cDNA itself. Indeed, all vectors intended for molecular interaction studies are designed in this fashion. FIG. 2 shows a typical example of such a vector for the current state of the art: The vector, known as JG4-5, is designed for two-hybrid screening of cDNA libraries using baker's yeast as host cells. The vector comprises an origin of replication for maintenance in bacterial cells, an antibiotic resistance gene, for selection in same, a second origin of replication for yeast, and a nutritional gene for selection in same. The vector further comprises a transcriptional control start signal and stop signal for expression of the hybrid gene, sequences encoding the common peptide including a translational start codon and a multiple cloning site or MCS for insertion of the cDNA.

A major shortcoming of vectors such as JG4-5 is illustrated in Edwards et al., (1997) Development 124: 3855-3864. Edwards et al. shows that amino acid 25 of the protein Tube is necessary for it to interact with the protein Pelle. However, two-hybrid screens using hybrid proteins derived from traditional vectors with the common peptide on the 5′ end fail to detect this interaction. This failure likely occurs because few or none of the cDNA inserts contain enough of the 5′ end of the Tube sequence to encode amino acid 25. The few cDNA inserts that do contain the 5′ region likely also contain a stop codon located only 75 base pairs before the sequence encoding amino acid 25 and thus result in a truncated hybrid protein that also lacks Tube amino acid 25. Absent a domain mapping study, current practical methods are unable to detect which two-hybrid interaction negatives are, like the Tube/Pelle interaction, actually false negatives arising from insufficient presentation of a functional amino region of the test protein.

Only a few methods have been devised to overcome the above-mentioned paucity of cDNAs representing the 5′ region of the mRNA. One approach, exemplified by U.S. Pat. No. 6,083,727 to Guegler, et. al (2000), involves enriching the library for clones containing the 5′ end of mRNAs. A second approach is to purify cDNAs that are full-length; that is, those which are complete copies of the initial mRNA molecules, as in U.S. Pat. No. 5,891,637 to Ruppert (1999) and U.S. Pat. No. 5,846,721 to Soares, et. al (1998). However, these methods are unusually demanding from a technical perspective and thus may prove prohibitively costly or time-consuming for widespread or high-throughput screens.

FIG. 3a shows that an mRNA molecule with a polyadenylated 3′ end can be reacted with synthetic oligonucleotides of random sequence which can anneal at various random locations along the length of the molecule. FIG. 3b shows that enzymatic first strand synthesis performed with primers of this nature results in a higher probability of reaching the 5′ end of the mRNA. This random-primed library therefore consists of a population of cDNAs differing in length at their 3′ ends but adequately representing the 5′ ends of the mRNAs.

Proper representation of the 5′ ends of mRNAs is widely regarded as a decided advantage for the construction of cDNA libraries. However, using current systems for molecular interaction methods, which place the common peptide at the amino terminus of the hybrid protein, it is not possible to fully exploit such libraries in which the 5′ ends are adequately represented because the 5′ cDNA region, including the UTR, would be placed at the 3′ end of the sequence encoding the common peptide. Because of positional effects within the hybrid protein, even though the 5′ region is expressed, it may not function simply because it is located at the wrong end of the hybrid protein.

A few vectors do currently exist which place the common peptide at the carboxyl terminus of the hybrid protein. In most cases, however, these vectors are intended for the expression of a known gene or gene fragment. Accordingly, they require knowledge of the nucleotide sequence of the gene or fragment for the design of cloning strategies that will result in proper expression. This is clearly not feasible for libraries composed of thousands of unknown sequences.

Other vectors of this type have been designed for library screens, although in these instances either the library or the screen is limited to a narrow range of applications. For example, phage display libraries sometimes encode the common peptide (a bacteriophage coat protein) at the carboxyl terminus, but said libraries are collections of small, synthetic oligonucleotides, all of which are present in equal proportions. This is not the case with cDNAs.

As another example, U.S. Pat. No. 6,103,472 to Thukral (2000), describes construction of a hybrid gene cDNA library with the cDNA encoded peptide at the amino terminus of the hybrid protein, but the library is specifically useful for detecting peptides with a single function, the ability to be secreted from within the cell. In order to be useful, hybrid gene cDNA libraries for molecular interaction methods must not be constrained by the nature of the insert. Further, since they are intended for use with various “baits”, each of which is expected to have a unique function, hybrid gene cDNA libraries for molecular interaction studies cannot be constrained by the function of the cDNA-encoded peptide.

Current cDNA vectors for molecular interaction methods, such as JG4-5, invariably place the common peptide at the amino-terminus of the hybrid protein. This places several constraints on the utility of the vectors and hybrid proteins during molecular interaction screening. Several significant constraints are as follows:

(a) The common peptide is expressed in the cells regardless of whether a cDNA insert is present in the vector. With certain methods of detection this may give rise to undesirable background signal.

(b) The common peptide determines the reading frame for the entire hybrid gene. Due to the random nature of the 5′ end of cDNAs, discrepancies in reading frame result in the production of hybrid peptides with unwanted, out-of-natural frame structures. This occurs in two thirds of all the clones in a library. Some are invariably detected as false positives.

(c) cDNAs that are copies of non-coding RNAs produce irrelevant hybrid peptides. As an example, ribosomal RNA or rRNA, which does not encode any proteins, is by far the most abundant RNA species in any cell. Consequently, even the most conscientiously prepared cDNA libraries can be expected to contain rRNA clones. With current vectors, these clones express rRNA hybrid proteins which can be detected as false positives. This occurs because the start codon is provided before the common peptide, so the lack of a start codon in most rRNA in no way prevents its expression.

(d) A substantial number of the already underrepresented fraction of cDNAs that do include the 5′ end of the corresponding mRNA contain an additional non-coding untranslated region or UTR found at 5′ end of the mRNAs. This precludes the possibility of generating productive hybrid proteins if the UTR separates the common peptide from the protein-coding region of the cDNA.

(e) In order to be functional, each protein must fold in a specific three-dimensional configuration. For individual segments or domains of proteins this is often dependent on the context in which they are found. For example, a protein modified so that its amino-terminal and carboxy-terminal portions are reversed will most often lose its function. Molecular interaction methods rely on the maintenance of domain function in the hybrid protein. Since current vectors for molecular interaction methods invariably place the cDNA downstream of the common peptide sequences, cDNA-derived protein domains that are intended to be amino-terminal are placed at the carboxyl-terminus of the hybrid protein. This can abolish function and result in false negative results.

### SUMMARY OF THE INVENTION

The invention includes a hybrid gene cDNA library comprising a series of vectors, each vector comprising a DNA molecule having at least one selectable marker sequence and a sequence encoding a hybrid protein region. The hybrid protein region comprises a regulatable sequence, a multiple cloning site that does not encode a translational termination sequence or a start codon placed immediately 3′ to the regulatable DNA sequence, a sequence encoding at least one common peptide and not containing a translation initiation codon placed 3′ to the multiple cloning site. Each vector of the library additionally comprises a single cDNA molecule inserted at the multiple cloning site. Each of these single cDNA molecules is obtained from a cDNA population generated using random primers. The vector is preferably a plasmid.

The vector may additionally comprise one or more origins of replication active in bacteria cells as well as one or more origins of replication active in yeast cells. The hybrid protein region may additionally comprise a DNA molecule which encodes a transcriptional termination sequence placed immediately 3′ to the DNA molecule encoding at least one common peptide.

In a more preferred embodiment, the regulatable sequence is the rat Glucocorticoid Response Element. In another preferred embodiment it may be an Estrogen Response Element. The common peptide is preferably encoded by a DNA molecule comprising sequences encoding all or portions of the GAL4 yeast transcriptional activator and six successive histidine residues or, alternatively, a nuclear localization sequence from the SV40 virus.

In one particular embodiment, the common peptide is encoded by a DNA molecule comprising sequences encoding an immunological epitope from adenoviral hemagluttinin.

The vector may also include one or more origins of replication active in yeast cells and one or more origins of replication active in bacterial cells. At least one yeast origin of replication is derived from the natural 2-micron yeast plasmid. The selectable marker sequences may be the bacterial ampicillin resistance gene and the yeast TRP 1 nutritional auxotrophy gene or, alternatively, the bacterial kanamycin resistance gene and the yeast URA3 nutritional auxotrophy gene. The preferred transcriptional termination sequence is derived from the yeast ADH 1 gene.

The present invention also includes a method of producing hybrid proteins. In this method, first a purified sample of a vector comprising a DNA molecule with at least one selectable marker sequence and a sequence encoding a hybrid protein region is provided. The hybrid protein region ideally comprises a regulatable DNA sequence, a multiple cloning site that does not encode a translational termination sequence placed immediately 3′ to the regulatable DNA sequence, and a DNA sequence encoding at least one common peptide and not containing a translation initiation codon placed 3′ to the multiple cloning site. Next, a mRNA template population of interest is isolated and a cDNA population is synthesized from the mRNA template population using random sequence oligonucleotide primers. This synthesis is preferably conducted using PCR. Cloning linkers may then be added to the cDNA population and it may be inserted into the vector, which has been cleaved at the multiple cloning site, thus creating a hybrid gene cDNA library. This library may then be expanded by transforming bacterial cells with the library and selecting then growing transformed cells. The library may then be purified from the transformed cells. In a preferred embodiment, the bacterial cells transformed with the hybrid gene cDNA library are E. coli cells.

The invention additionally includes a method of performing a yeast two-hybrid assay. First a hybrid gene cDNA library of the present invention is provided in which the common peptide includes a DNA activation domain. The library is then used to transform yeast cells which contain another hybrid protein. This other hybrid protein includes a DNA binding polypeptide and a bait polypeptide as well as a DNA molecule with a sequence to which the DNA binding polypeptide may bind. In the vicinity of this sequence the DNA molecule also contains a sequence activatable by the DNA activation domain of the cDNA library hybrid protein. The DNA molecule additionally includes a reporter sequence that may be activated if the DNA activation domain is brought into proximity with the activatable sequence. Transformed cells are then selected and an assay may be performed to detect activation of the reporter sequence. Activation is indicative that the polypeptide encoded by the particular cDNA insert in a given cell is capable of interaction with the bait polypeptide.

In a preferred embodiment of this method, the DNA activation domain is derived from the yeast the GAL 4 activation domain, and the reporter sequence is derived from the yeast GAL 4 gene. Additionally, the hybrid gene cDNA library vector preferably includes a TRP 1 nutritional auxotrophy gene as the selectable marker sequence and the yeast cells are trp 1 mutant yeast cells. Alternatively, the vector may include a URA 3 nutritional auxotrophy gene as the selectable marker sequence and the yeast cells may be ura 3 mutant yeast cells.

In still another preferred embodiment, the common peptide may additionally comprise a nuclear localization sequence which may be the nuclear localization sequence from the SV40 virus.

Accordingly, several objects and advantages of the present invention are:

(a) to eliminate the potential background and false positives resulting from vectors that lack a cDNA insert.

(b) to eliminate hybrid proteins derived from reading frame shifts in the cDNA-derived protein segment of the hybrid-protein relative to the common peptide.

(c) to eliminate hybrid proteins resulting from the presence of cDNAs from noncoding RNAs, such as mRNAs.

(d) to avoid the disruption of reading frame continuity by the presence of 5′ UTRs in the cDNA.

(e) to place the amino-terminal peptide domains from the cDNA library at the amino-terminus of the hybrid protein.

Further objects and advantages are to provide a method for the construction of hybrid gene cDNA libraries that is simple and efficient, yet allows the cloning of cDNAs that represent the 5′ region of the starting mRNAs, and that is not constrained either by the nature of the inserts or by the function of the peptides encoded therein. Still further objects and advantages will become apparent from a consideration of the Detailed Description and Drawings. It will be understood by one skilled in the art that every embodiment of the present invention need not necessarily fulfill all objects and advantages of the overall invention. A more detailed understanding of the invention may be had through reference to the Detailed Description.

### BRIEF DESCRIPTION OF THE DRAWINGS

FIG. 1 illustrates the method and results of oligo[dT]-primed cDNA synthesis, with a population of cDNAs.

FIG. 1a shows the oligo[dT]-primer annealed to the poly-A tail of the RNA.

FIG. 1b shows the various lengths of cDNA molecules obtained before reverse transcriptase falls off the RNA. As the three example cDNAs indicate, this method is biased towards representation of the 3′ end of the RNA.

FIG. 2 is a diagram of JG4-5, a current state of the art vector for the construction of hybrid gene cDNA libraries, with the DNA sequences encoding the common peptide 5′ to the multiple cloning site.

FIG. 3 illustrates the method and results of random-primed cDNA synthesis with a population of cDNAs.

FIG. 3a shows the random primers annealed to random sequence at various locations along the RNA.

FIG. 3b shows various lengths of cDNA molecules obtained before reverse transcriptase falls off the RNA. As the three example cDNAs indicate, this method is not biased towards any portion of the RNA so the 5′ end is represented as well as other regions.

FIG. 4 is a diagram of one embodiment of the present invention, with the DNA sequences encoding the common peptide 3′ to the multiple cloning site.

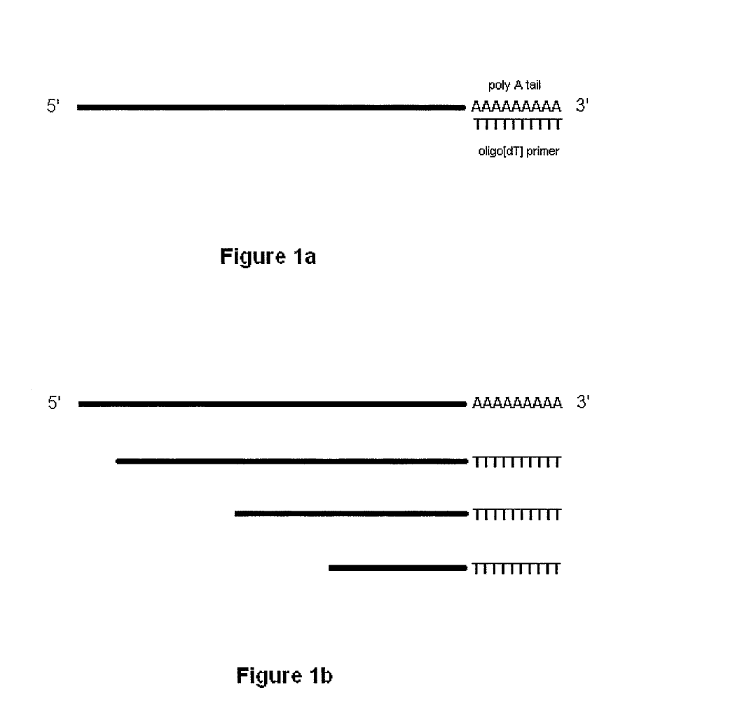

### DETAILED DESCRIPTION OF THE INVENTION

The present invention provides hybrid gene cDNA libraries. It also provides methods for using such libraries to allow the cloning and detection, as hybrid genes or hybrid proteins, of sequences that encode functional amino-terminal peptides from the 5′ end of mRNAs.

The vectors of the present invention used in construction of the hybrid cDNA libraries generally have one or more origin(s) of replication to allow for replication and/or maintenance in yeast or bacteria cells, if the vector is to be used in such cells, a selectable marker sequence allowing selection of cells comprising the vector, and a sequence encoding a hybrid protein region. The sequence encoding a hybrid protein region comprises a regulatable DNA sequence, a multiple cloning site (MCS) placed immediately downstream, or 3′ to the regulatable DNA sequence that does not contain translational termination sequences, and sequences encoding at least one common peptide, but not encoding a translation initiation codon located downstream, or 3′ to the MCS. Immediately 3′ or downstream of the common protein sequence a transcriptional termination sequence may be included to ensure proper termination and processing of the hybrid gene mRNA.

In a preferred embodiment, the regulatable DNA sequence in the hybrid protein region is the Glucocorticoid Response Element (GRE) from rat and the common peptide is encoded by a fusion of sequences derived from the DNA binding domain of the yeast transcriptional activator GAL4 and sequences encoding six successive histidine residues. The GAL 4 sequences make the hybrid fusion protein useful in yeast two-hybrid assays and the histidine sequences are useful for affinity purification of the hybrid protein.

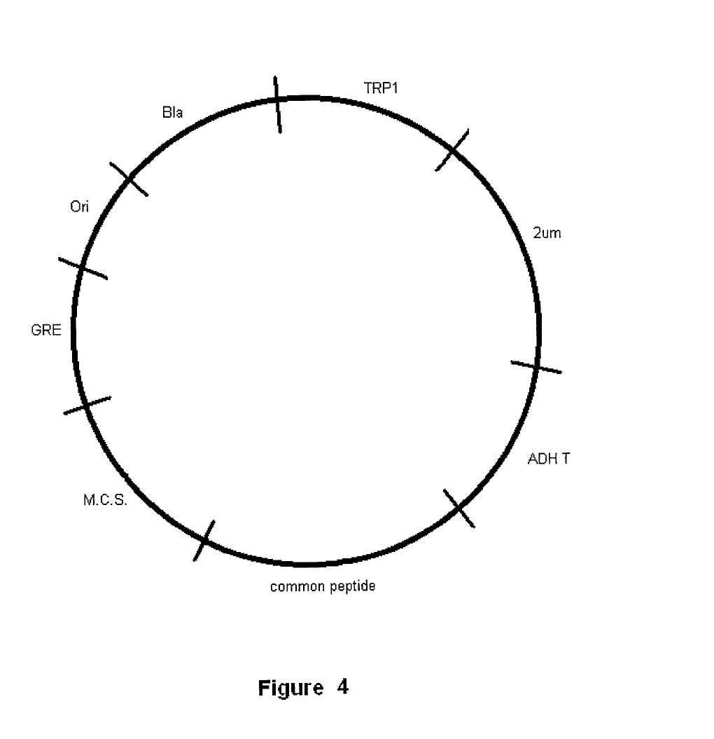

Additionally, the vector preferably contains both a bacterial origin of replication and a yeast origin of replication, in particular, an origin of replication derived from the natural 2-micron yeast plasmid. The vector also comprises a bacterial ampicillin resistance gene for propagation and selection in E. coli, and the yeast TRP 1 nutritional auxotrophy gene for propagation and selection in trp 1 mutant yeast. This preferred embodiment is depicted in FIG. 4.

In other preferred embodiments, the selectable marker is a bacterial antibiotic resistance gene conferring resistance to kanamycin and the yeast nutritional auxotrophy gene is URA3, which confers upon ura3 mutant yeast the ability to grow in the absence of supplemental uracil. The nucleotide sequences encoding a common peptide may be derived from the GAL4 activation domain fused to a nuclear localization sequence from the virus SV40, also for use in a yeast two-hybrid assay. The common peptide sequences may also be sequences encoding an immunological epitope from adenoviral hemagluttinin. The DNA regulatory sequence may be an Estrogen Response Element.

There are various possibilities with regard to the disposition of certain elements which constitute the vector, as their relative placement and orientation do not affect its performance. This applies to both origins of replication and both selectable marker genes as to their placement relative to each other, and to their collective placement on either side of hybrid protein region. Only the hybrid protein region is intended to have the internal disposition of elements described above.

Other alternative embodiments result from the substitution of one or more of any of the elements by other similar elements which may serve a similarly useful function. For example, different origins of replication and/or selectable markers suitable for other host cells may be useful as may different transcriptional initiation and/or termination sequences, multiple cloning sites designed for specific applications, and sequences encoding common peptides with different detectable functions. These functions may be suitable for molecular interaction methods but are not limited to these methods, and alternative embodiments of the present invention can be designed to suit other specific applications of hybrid gene libraries.

In the hybrid gene cDNA library, multiple copies of the vector are present and each vector contains a cDNA insert at the multiple cloning site. The hybrid gene cDNA library may be generated using the vector described above and any insertion techniques known to the art. However, the cDNA molecules which are inserted into the vector to form the cDNA library are preferably obtained using random primers as described below.

The method of preparing the hybrid gene cDNA library of the present invention may comprise a number of steps, each of which can be readily performed in any laboratory with the equipment and skills in the art. Specifically, for the embodiment depicted in FIG. 4 and similar embodiments the steps include:

(a) Propagation of the vector in E. coli cells, and purification of vector DNA;

(b) Isolation or acquisition of the mRNA template population of interest, and synthesis of a cDNA population from the template using random sequence oligonucleotide primers;

(c) Addition of cloning linkers to the cDNA population and insertion of the cDNA into the appropriately cleaved vector (e.g. cleaved at the MCS);

(d) Transformation of Escherichia coli cells with the hybrid gene cDNA library, and propagation and purification of same;

(e) Transformation of yeast cells, selection for transformed cells and performance of yeast two-hybrid screen.

(f) Identification, purification and propagation of positive clones.

(g) Affinity purification of hybrid protein via the 6×-Histidine tag.

The precise details of each of the above steps can be modified to suit individual applications and embodiments of the invention.

As this description makes clear, the present invention avoids several of the shortcomings of previous vectors. First, the vector of the invention will not express the common peptide unless it contains a cDNA insert. Because the vector relies on the cDNA's own start codon and not one placed before the common peptide or before the cDNA insert, as in the prior art, no common peptide may be produced by any vector that does not contain a cDNA insert comprising a start codon. Therefore, the vector of the present invention is incapable of producing the common peptide unless it is part of a hybrid protein, thereby avoiding background signal in may types of assays.

Second, hybrid proteins cannot contain an out of frame polypeptide encoded by the cDNA insert because the insert itself comprises the start codon and determines the reading frame. In many previous vectors the cDNA may be translated in frame with the common peptide, but often out of its natural reading frame. These out-of-natural frame regions may interact with molecules with which the natural, in-frame peptide will not interact, thus giving false positives in a molecular interaction screening. In the present invention, the cDNA-generated polypeptide is always in frame. The common peptide may be out of frame in two thirds of the hybrid proteins, but, because the sequence of the common peptide is known, the amino acid sequence of out-of-frame common peptides may be determined. If the out-of-frame common peptides are likely to cause false results or otherwise interfere with an assay using the hybrid proteins, steps may be taken to avoid this by using a different common peptide or to detect false results.

Third, with previously known vectors, hybrid proteins comprising a common peptide and a peptide encoded by ribosomal RNA are common. These peptides may produce high background levels in many assays or even false positives. This problem is avoided in the present invention because it is very unlikely that a vector with a rRNA-derived cDNA will be able to produce a hybrid protein comprising the common peptide. Most rRNA derived cDNAs will lack a start codon. Additionally, rRNA is replete with stop codons, so it is unlikely translation will progress for enough to reach the common peptide sequence.

Fourth, most previous vectors for use with a hybrid gene cDNA library seriously underrepresent the 5′ end of RNAs. Essentially, even if cDNA generated using random primers so the 5′ ends are represented, these 5′ ends often contain a portion of the 5′ untranslated region. As shown in Edwards et al. and described more fully in the Background, this untranslated region may encode stop codons or other sequences that interfere with translation or folding or stability of the translated protein. Using conventional vectors with the cDNA placed 3′ relative to the common peptide DNA, the 5′ untranslated region generally interferes with translation and precludes representation of the 5′ ends of RNAs. Thus interactions, such as those between Tube an Pelle which are virtually undetectable with present techniques may be readily observed using a hybrid gene cDNA library of the present invention.

In the present invention, the 5′ UTR is of no relevance to translation of a complete hybrid protein because it is 5′ relative to the start codon. Essentially, by placing the 5′ UTR in a more natural position, the present invention abrogates its ability to interfere with translation of the hybrid protein.

Fifth, the 5′ end of RNA usually encodes for the amino terminus of a protein. However, in previous vectors this normally amino terminal region is placed on the carboxy terminus of the hybrid protein. This placement may interfere with the three-dimensional structure and domain function of the peptide encoded by the 5′ RNA region, rendering it unable to interact with other proteins in a normal manner. As a result, many false negatives may be obtained if such hybrid proteins are used in molecular interaction studies. The present invention avoids this problem by placing the 5′ end of the RNA via the cDNA in the 5′ portion of the hybrid gene. Therefore amino terminal domains are located in the amino terminus of the hybrid protein and are more likely to retain their normal three-dimensional structures and functions.

The present invention has application in many circumstances. One important application is in any assay or study in which one wishes to detect all of a particular type of molecular interaction, such as all proteins in a cell capable of interacting with another protein. To avoid positional effects resulting from the 3′ end of the RNA being placed at the 5′ end of the hybrid protein, the vector library of the present invention may be combined with a more traditional vector library. Situations in which this method is desirable to detect all interactions and ways in which multiple types of hybrid gene libraries may be combined in studies will be apparent to one skilled in the art.

In order to facilitate a more complete understanding of the invention, a number of Examples are provided below. However, the scope of the invention is not limited to specific embodiments disclosed in these Examples, which are for purposes of illustration only. Some alternative embodiments are described above and others will be apparent to those skilled in the art.

### EXAMPLES

### Example 1

### GAL4/Histidine Common Peptide Hybrid Gene Library Vector

One preferred embodiment of the vector of the present invention is depicted in FIG. 4. The vector is a circular DNA molecule comprising a bacterial origin of replication and the bacterial ampicillin resistance gene Bla for propagation and manipulation in Escherichia coli cells. The vector further comprises the yeast TRP1 nutritional auxotrophy gene for vector selection in trp1 mutant yeast and a yeast origin of replication derived from the natural 2-micron yeast plasmid. Expression of the hybrid protein is driven by a regulatable DNA sequence, related to the Glucocorticoid Response Element GRE from rat. A multiple cloning site for ligation of the cDNA inserts is placed immediately adjacent to and in a 3′ or downstream orientation to the GRE. The multiple cloning site is designed to not contain the translational termination sequences TAA, TAG or TGA in any reading frame. Adjacent to and 3′ or downstream of the multiple cloning site are sequences encoding the common peptide which is itself a fusion of sequences derived from the DNA binding domain of the yeast transcriptional activator GAL4 and sequences encoding six successive histidine residues for affinity purification of the hybrid protein. Notably, the sequences in the common peptide lack a translational initiation codon. Finally, adjacent and in a 3′ orientation or downstream of the common peptide sequences is a transcriptional terminator derived from the yeast ADH1 gene to ensure proper termination of transcription and processing of the hybrid gene mRNA. The region comprising the DNA regulatory element, MCS, common peptide, and transcriptional terminator is known as the hybrid protein region.

### Example 2

### Method of Producing and Purifying Hybrid Protein Products

A method of using the vector described in Example 1 consists of a number of steps, each of which can be readily performed in any laboratory with the equipment and skills in the art. Specifically, for the embodiment depicted in FIG. 4 and described in Example 1 the steps are:

(a) Propagation of the vector in Escherichia coli cells, and purification of vector DNA.

(b) Isolation or acquisition of the mRNA template population of interest, and synthesis of a cDNA population using random sequence oligonucleotide primers.

(c) Addition of cloning linkers to the cDNA population and insertion of a single molecule of the cDNA into an appropriately cleaved vector. This occurs in multiple vectors simultaneously so that nearly all of the cDNA molecules are each inserted into a separate vector.

(d) Transformation of Escherichia coli cells with the hybrid gene cDNA library, and propagation and purification of same.

(e) Transformation of yeast cells and performance of yeast two-hybrid screen.

(f) Identification, purification and propagation of positive clones.

(g) Affinity purification of hybrid protein via the 6×-Histidine tag.

### Example 3

### Hybrid Gene Library Screen

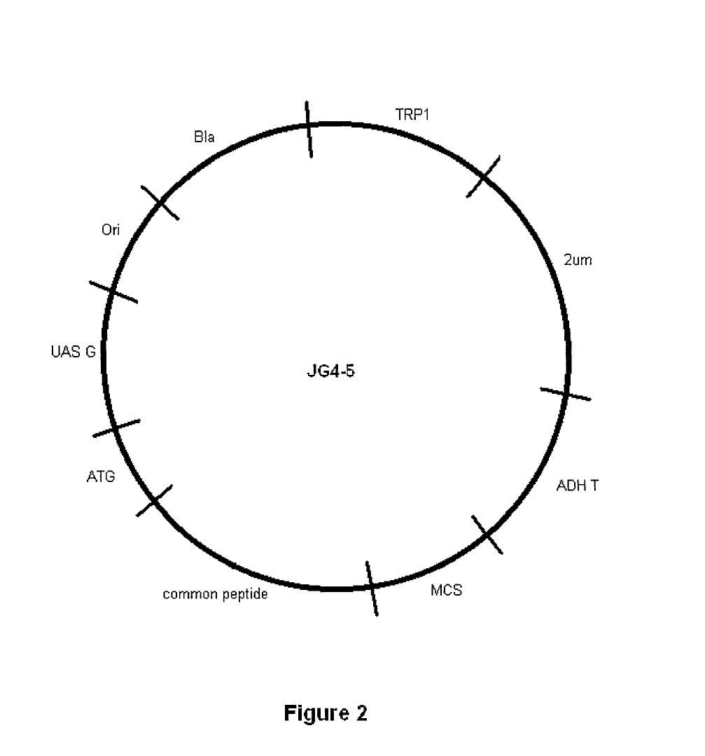

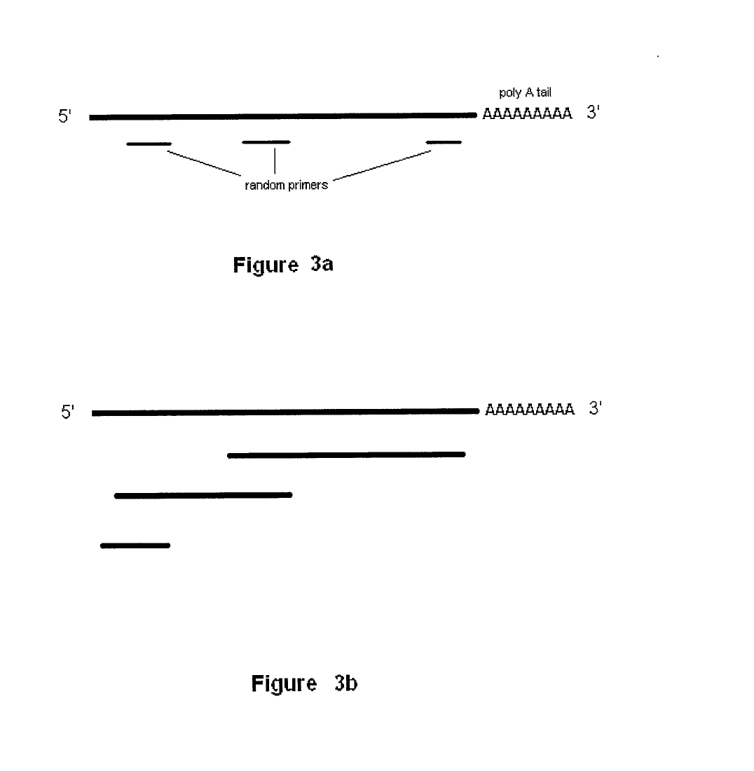

A cDNA population derived from of a cell known to express Tube has been prepared and inserted into the JG4-5 vector of FIG. 2. The common peptide is a polypeptide derived from the GAL 4 activation domain, but it may also be a different transcriptional activator. The resulting hybrid gene cDNA library is then be used in a standard yeast two-hybrid assay by transforming yeast in which a hybrid protein comprising the Pelle bait polypeptide and a DNA binding polypeptide is also present. The reporter sequence in such an assay is derived from the yeast β-gal gene. The cDNA sequences of interacting hybrid proteins which activate the reporter sequence and yield positive results in the assay were then analyzed. As shown in previous studies in Edwards et al., no positives are observed.

The same cDNA population has also been placed in the vector of this invention, shown in FIG. 4, in which the common peptide is the same as in the JG4-5 vector, and subjected to the same two-hybrid assay. In this assay true positives are observed. Analysis confirms that they represent vectors comprising the 5′ RNA sequence of Tube, which encodes amino acid 25. Thus, the identical two-hybrid assay using the vector of this invention with a cDNA population generated according to the invention uncover an interaction not detected using a conventional vector and a polyA-generated cDNA population.

I claim:

1. A hybrid gene cDNA library comprising a series of vectors, each vector comprising a DNA molecule having at least one selectable marker sequence and a sequence encoding a hybrid protein region, wherein the hybrid protein region comprises,
a) a regulatable DNA sequence,
b) a multiple cloning site immediately 3′ to the regulatable DNA sequence, wherein the multiple cloning site does not encode a translational termination sequence, and
c) a DNA sequence encoding at least one common peptide placed 3′ to the multiple cloning site, wherein the common peptide encoding sequence does not contain a translation initiation codon, and wherein the each vector of the library additionally comprises a single cDNA molecule inserted at the multiple cloning site wherein each of said cDNA molecules is obtained from a cDNA population generated using random primers.

2 The hybrid gene cDNA library of claim 1 wherein each vector additionally comprises one or more origins of replication active in bacteria cells.

3. The hybrid gene cDNA library of claim 1, wherein each vector additionally comprises one or more origins of replication active in yeast cells.

4. The hybrid gene cDNA library of claim 1, wherein the hybrid protein region additionally comprises a sequence which encodes a transcriptional termination sequence placed immediately 3′ to the DNA sequence encoding the at least one common peptide.

5. The hybrid gene cDNA library of claim 1, wherein the regulatable DNA sequence is the rat Glucocorticoid Response Element

6. The hybrid gene cDNA library of claim 1, wherein the regulatable DNA sequence is an Estrogen Response Element.

7. The hybrid gene cDNA library of claim 1, wherein the common peptide is encoded by a DNA molecule comprising sequences encoding all or portions of the GAL4 yeast transcriptional activator and six successive histidine residues.

8. The hybrid gene cDNA library of claim 1, wherein the common peptide is encoded by a DNA molecule comprising sequences encoding all or portions of the GAL4 yeast transcriptional activator and a nuclear localization sequence from the SV40 virus.

9. The hybrid gene cDNA library of claim 1, wherein the common peptide is encoded by a DNA molecule comprising sequences encoding an immunological epitope from adenoviral hemagluttinin.

10. The hybrid gene cDNA library of claim 1, wherein each of the vectors additionally comprises one or more origins of replication active in yeast cells and one or more origins of replication active in bacterial cells, wherein at least one yeast origin of replication is derived from the natural 2-micron yeast plasmid.

11. The hybrid gene cDNA library of claim 1, wherein the selectable marker sequences are selected from the group consisting of the bacterial ampicillin resistance gene and the yeast TRP 1 nutritional auxotrophy gene.

12. The hybrid gene cDNA library of claim 1, wherein the selectable marker sequences are selected from the group consisting of the bacterial kanamycin resistance gene and the yeast URA3 nutritional auxotrophy gene.

13. The hybrid gene cDNA library of claim 4, wherein the transcriptional termination sequence is derived from the yeast ADH 1 gene.

14. A method of producing hybrid proteins from a hybrid gene cDNA library comprising:
a) providing a purified sample of a vector comprising a DNA molecule having at least one selectable marker sequence and a sequence encoding a hybrid protein region, wherein the hybrid protein region comprises,
i) a regulatable DNA sequence,
ii) a multiple cloning site immediately 3′ to the regulatable DNA sequence, wherein the multiple cloning site does not encode a translational termination sequence, and
iii) a DNA sequence encoding at least one common peptide placed 3′ to the multiple cloning site, wherein the common peptide encoding sequence does not contain a translation initiation codon,
b) isolating a mRNA template population of interest;
c) synthesizing a cDNA population from the mRNA template population using random sequence oligonucleotide primers;
d) adding cloning linkers to the cDNA population,
e) cleaving the vectors at the multiple cloning site,
f) inserting the cDNA population molecules into the cleaved vectors, to create a hybrid gene cDNA library,
g) transforming bacterial cells with the hybrid gene cDNA library and selecting transformed cells,
h) purifying the hybrid gene cDNA library from the transformed bacterial cells;
i) transforming yeast cells with the hybrid gene cDNA library and selecting transformed cells, and
j) allowing transformed yeast cells to produce a hybrid protein.

15. The method of claim 14, wherein the bacterial cells transformed with the hybrid gene cDNA library are E. coli cells.

16. The method of claim 14, wherein the vector encodes a common peptide sequence comprising six successive histidine residues and the hybrid protein is purified from the yeast cells using affinity purification.

17. A method of performing a yeast two-hybrid assay comprising:
a) providing a hybrid gene cDNA library comprising a series of vectors, each vector comprising a DNA molecule having at least one selectable marker sequence and a sequence encoding a hybrid protein region, wherein the hybrid protein region comprises,
i) a regulatable DNA sequence,
ii) a multiple cloning site immediately 3′ to the regulatable DNA sequence, wherein the multiple cloning site does not encode a translational termination sequence, and
iii) a DNA sequence encoding at least one common peptide placed 3′ to the multiple cloning site, wherein the common peptide encoding sequence does not contain a translation initiation codon, and wherein the each vector of the library additionally comprises a single cDNA molecule inserted at the multiple cloning site wherein each of said cDNA molecules is obtained from a cDNA population generated using random primers,
wherein the common peptide comprises a DNA activation domain,
b) providing yeast cells comprising a second hybrid protein comprising a DNA binding polypeptide and a bait polypeptide and additionally comprising a DNA molecule comprising a sequence to which the DNA binding polypeptide may bind, a sequence activatable by the DNA activation domain and a reporter sequence,
c) transforming the yeast cells with the hybrid gene cDNA library and selecting transformed yeast cells, and
d) performing an assay to detect activation of the reporter sequence.

18. The method of claim 17 wherein the DNA activation domain is derived from the yeast the GAL 4 activation domain, and the reporter sequence is derived from the yeast GAL 4 gene.

19. The method of claim 17, wherein the vector comprises a TRP 1 nutritional auxotrophy gene as the selectable marker sequence and the yeast cells are trp 1 mutant yeast cells.

20. The method of claim 17, wherein the vector comprises a URA 3 nutritional auxotrophy gene as the selectable marker sequence and the yeast cells are ura 3 mutant yeast cells.

21. The method of claim 17, wherein the common peptide additionally comprises a nuclear localization sequence.

22. The method of claim 21, wherein the nuclear localization sequence is a nuclear localization sequence from the SV40 virus.
